(19) United States
(12) Patent Application Publication
(10) Pub. No.: US 2009/0247938 A1
(43) Pub. Date: Oct. 1, 2009

(54) Intraoperative hypotony mitigation

(76) Inventor: David C. Buboltz, Upland, CA (US)

Correspondence Address: ALCON, IP LEGAL, TB4-8, 6201 SOUTH FREEWAY, FORT WORTH, TX, 76134, US

(21) Appl. No.: 12/079,812

(22) Filed: Mar. 28, 2008

(51) Int. Cl. A61F 9/007

(52) U.S. Cl. 604/31; 604/30; 606/166

(57) ABSTRACT

A method of mitigating an intraoperative hypotony condition using experimental calibrations of different infusion cannulas and vitrectomy probes showing the relationship between infusion pressure, cut rate, and intraocular pressure for various levels of aspiration vacuum in an ophthalmic surgical system.

### FIELD OF THE INVENTION

The present invention generally pertains to microsurgical systems and more particularly to mitigating hypotony in ophthalmic surgery.

### DESCRIPTION OF THE RELATED ART

During small incision surgery, and particularly during ophthalmic surgery, small probes are inserted into the operative site to cut, remove, or otherwise manipulate tissue. During vitrectomy surgery, fluid is typically infused into the eye via an infusion cannula, and the infusion fluid and eye tissue are typically aspirated from the surgical site via a vitrectomy probe.

Maintaining an optimum intraocular pressure (“IOP”) during vitrectomy surgery is currently problematic. Under certain conditions in vitrectomy surgery, the flow out of the eye through the vitrectomy probe exceeds the flow into the eye from the infusion cannula. Such conditions may create a low or negative intraocular pressure. Intraocular pressure below 6 mmHg is deemed hypotony. Hypotony may cause serious complications for the patient such and retinal detachment or choroidal hemorrhage.

Accordingly, a need continues to exist for an improved method of controlling intraocular pressure during ophthalmic surgery and guarding against hypotony in particular.

### SUMMARY OF THE INVENTION

In one aspect, the present invention is a method of mitigating hypotony during ophthalmic surgery. A calibration of cut rate, infusion pressure, and intraocular pressure is created for selected aspiration vacuums using a selected ophthalmic surgical system, infusion cannula, and vitrectomy probe. The calibration is stored in a computer operatively coupled to the ophthalmic surgical system. The infusion cannula is used to provide surgical irrigating fluid to an eye. The vitrectomy probe is used to cut eye tissue and aspirate the irrigating fluid and the eye tissue. Actual values of the cut rate, the infusion pressure, and the aspiration vacuum are compared to the calibration using the computer to predict a hypotony condition. A warning signal is sent to a user of the ophthalmic surgical system via the computer and a user interface operatively coupled to the computer in response to the comparing step.

### BRIEF DESCRIPTION OF THE DRAWINGS

For a more complete understanding of the present invention, and for further objects and advantages thereof, reference is made to the following description taken in conjunction with the accompanying drawings, in which:

FIG. 1 is a schematic diagram illustrating infusion and aspiration in an ophthalmic surgical system; and

FIGS. 2A-C are exemplary calibrations of different infusion cannulas and vitrectomy probes showing the relationship between infusion pressure, cut rate, and intraocular pressure for various levels of aspiration vacuum of the ophthalmic surgical system of FIG. 1.

### DETAILED DESCRIPTION OF THE PREFERRED EMBODIMENTS

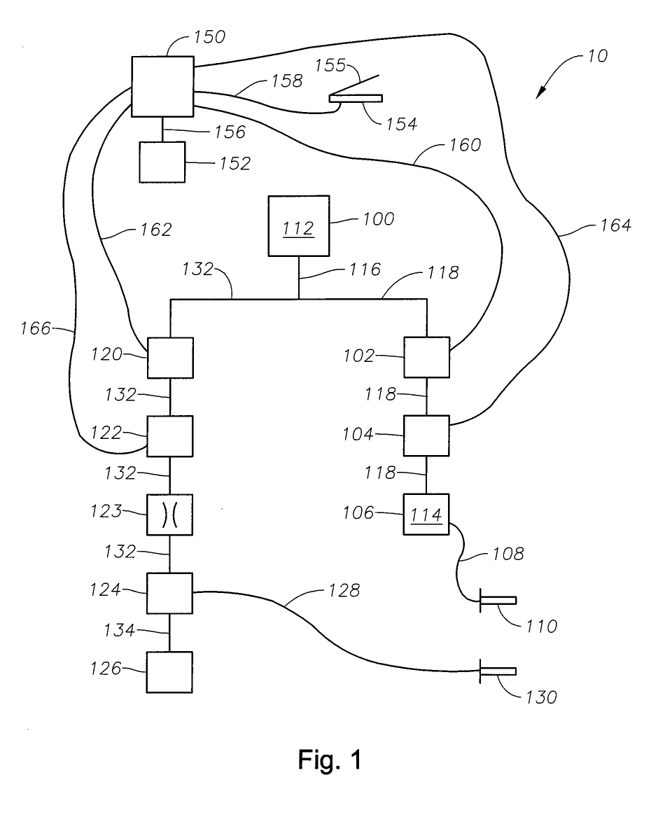

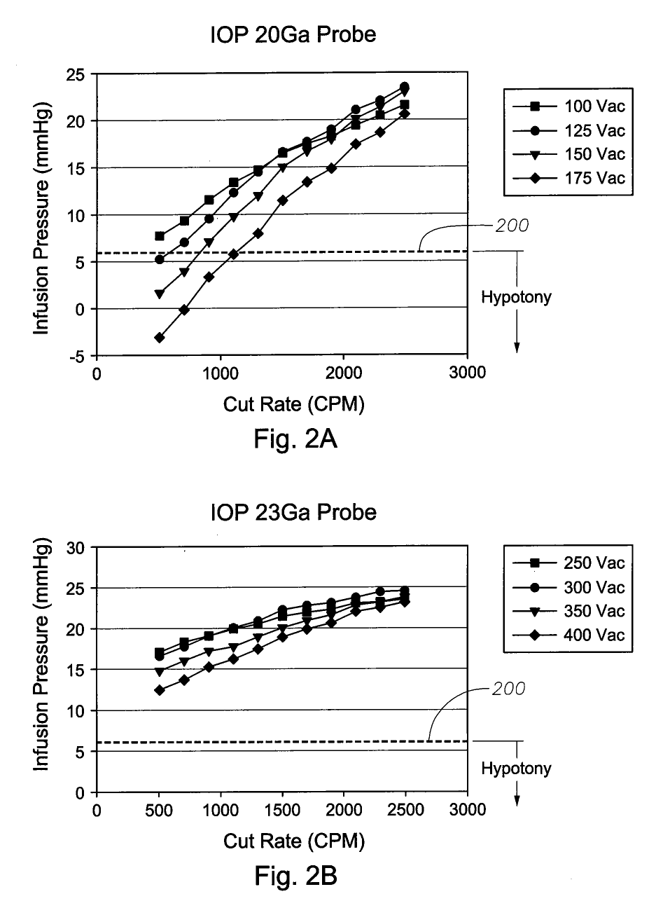

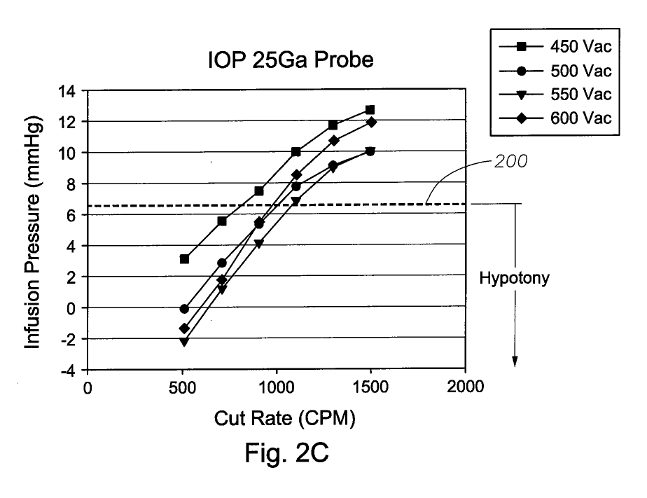

The preferred embodiments of the present invention and their advantages are best understood by referring to FIGS. 1-2C of the drawings, like numerals being used for like and corresponding parts of the various drawings. As shown in FIG. 1, ophthalmic surgical system 10 generally includes a pressurized gas source 100, an “on/off” solenoid valve 102, a proportional solenoid valve 104, an infusion fluid source 106, flexible tubing 108, and an infusion cannula 110. Pressurized gas source 100 preferably contains pressurized air 112. Infusion fluid source 106 holds a surgical irrigating fluid 114 such as, by way of example, BSS PLUS® intraocular irrigating solution available from Alcon Laboratories, Inc. of Fort Worth, Tex. Pressurized gas source 100 is fluidly coupled to infusion fluid source 106 via gas lines 116 and 118. Flexible tubing 108 fluidly couples infusion fluid source 106 to infusion cannula 110.

Surgical system 10 generally also includes an “on/off” solenoid valve 120, a proportional solenoid valve 122, a vacuum generator 123, a surgical cassette 124, a collection bag 126, flexible tubing 128, and a vitrectomy probe 130. Pressurized gas source 100 is fluidly coupled to vacuum generator 123 and surgical cassette 124 via a gas line 132. Flexible tubing 128 fluidly couples surgical cassette 124 and vitrectomy probe 130. A fluid line 134 fluidly couples surgical cassette 124 and collection bag 126. Vacuum generator 123 may be any suitable device for generating vacuum but is preferably a vacuum chip or venturi chip that generates vacuum when pressurized air 112 is passed therethrough. Vitrectomy probe 128 is capable of cutting and aspirating ophthalmic tissue into surgical cassette 124 and collection bag 126.

Surgical system 10 generally also includes a computer or microprocessor 150, a user interface 152, and a proportional control device 154. User interface 152 is preferably a graphical user interface having an audio speaker, and most preferably includes a touch screen display. Proportional control device 154 may be any device suitable for proportionally controlling surgical system 10, but is preferably a foot controller having a movable pedal or treadle 155. Computer 150, user interface 152, proportional control device 154, “on/off” solenoid values 102 and 120, and proportional solenoid valves 104 and 122 are electrically coupled via interfaces 156, 158, 160, 162, 164, and 166, respectively.

FIGS. 2A-C illustrate exemplary, experimentally determined calibrations of different sizes of vitrectomy probes and infusion cannulas used during operation of surgical system 10 showing the relationship between infusion pressure, cut rate, and intraocular pressure for various levels of aspiration vacuum. FIG. 2A shows such calibration for a 20 Ga infusion cannula 110 and 20 Ga vitrectomy probe 130. FIG. 2B shows such calibration for a 23 Ga infusion cannula 110 and 23 Ga vitrectomy probe 130. FIG. 2C shows such calibration for a 25 Ga infusion cannula 110 and 25 Ga vitrectomy probe 130. A hypotony condition is indicated via lines 200 in FIGS. 2A-C. Such calibrations are preferably stored as data lookup tables in computer 150. Of course, such calibrations may be determined for different surgical systems 10, different infusion cannulas and/or vitrectomy probes, and/or different levels of aspiration vacuum. As shown in FIG. 2B, a hypotony condition is not anticipated for the calibration for the particular 23 Ga infusion cannula and 23 Ga vitrectomy probe used with surgical system 10.

During a vitrectomy procedure, a nurse initiates priming of tubing 108 with surgical irrigating fluid 114 from infusion fluid source 106 via user interface 152. Computer 150 sends appropriate signals to open on/off solenoid valve 102 and proportional solenoid valve 104 to pressurize infusion fluid source 106 to accomplish priming. A surgeon sets the desired aspiration vacuum via user interface 152, and the surgeon uses proportional control device 154 to proportionally vary cut rate of vitrectomy probe 130 and the infusion pressure to infusion cannula 110 in response to the position of the control device 154. Preferably, the surgeon uses a foot controller 154 to proportionally vary cut rate of vitrectomy probe 130 and the infusion pressure to infusion cannula 110 in response to the position of pedal 155. Variation of the cut rate may be accomplished via conventional pneumatic and electrical means. Variation of the infusion pressure is accomplished via conventional pneumatic and electrical means such as computer 150 sending appropriate signals to proportional solenoid valve 104 in response to the position of proportional control device 154 (or pedal 155 of foot controller 154). Infusion cannula 110 thus provides surgical irrigating fluid to the eye in the conventional manner, and vitrectomy probe 130 cuts and aspirates surgical irrigating fluid and ophthalmic tissue in the conventional manner.

During such operation, computer 150 predicts hypotony by comparing the actual cut rate, infusion pressure, and aspiration vacuum to the appropriate data look up table illustrated in FIGS. 2A-C. If the intraocular pressure is approaching line 200 (6 mmHg) in the appropriate FIG. 2A-2C, computer 150 initiates an appropriate visual and/or audible warning signal to the surgeon via user interface 152. Alternatively, or in addition, computer 150 may require the surgeon to send a confirmation signal to computer 150 via user interface 152 or proportional control device 154 before continuing the vitrectomy procedure.

From the above, it may be appreciated that the present invention provides an improved method of controlling intraocular pressure during ophthalmic surgery and guarding against hypotony in particular. The present invention is illustrated herein by example, and various modifications may be made by a person of ordinary skill in the art. For example, while the present invention is described above relative to a vitrectomy probe, it is also applicable to an aspiration probe, such as, by way of example, a soft tipped cannula.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims

What is claimed is:

1. A method of mitigating hypotony during ophthalmic surgery, comprising the steps of:
creating a calibration of cut rate, infusion pressure, and intraocular pressure for selected aspiration vacuums using a selected ophthalmic surgical system, infusion cannula, and vitrectomy probe;
storing said calibration in a computer operatively coupled to said ophthalmic surgical system;
using said infusion cannula to provide surgical irrigating fluid to an eye;
using said vitrectomy probe to cut eye tissue and aspirate said irrigating fluid and said eye tissue;
comparing actual values of said cut rate, said infusion pressure, and said aspiration vacuum to said calibration using said computer to predict a hypotony condition; and
sending a warning signal to a user of said ophthalmic surgical system via said computer and a user interface operatively coupled to said computer in response to said comparing step.

2. The method of claim 1 further comprising the step of requiring said user to send a confirmation signal to said computer via said user interface before continuing said steps of using said infusion cannula and using said vitrectomy probe.

3. The method of claim 1 further comprising the step of creating a second calibration using a second selected infusion cannula and vitrectomy probe.

4. The method of claim 1 further comprising the step of varying said actual value of said cut rate of said vitrectomy probe and said actual value of said infusion pressure of said infusion cannula via a proportional control device.

5. The method of claim 4 further comprising the step of varying said actual value of said aspiration vacuum via a proportional control device.

6. The method of claim 1 wherein said warning signal is a visual signal.

7. The method of claim 1 wherein said warning signal is an audible signal.
